(19) United States
(12) Patent Application Publication
(10) Pub. No.: US 2014/0051635 A1
(43) Pub. Date: Feb. 20, 2014

(54) COMBINATION THERAPY FOR TREATMENT OF CANCER

(71) Applicant: Hung T. Khong, Mobile, AL (US)

(72) Inventor: Hung T. Khong, Mobile, AL (US)

(73) Assignee: UNIVERSITY OF SOUTH ALABAMA, Mobile, AL (US)

(21) Appl. No.: 13/982,743

(22) PCT Filed: Feb. 1, 2012
(86) PCT No.: PCT/US12/23530

§ 371 Date: Oct. 24, 2013

Related U.S. Application Data
(60) Provisional application No. 61/462,431, filed on Feb. 2, 2011.
(60) Provisional application No. 61/487,202, filed on May 17, 2011.

(51) Int. Cl. A61K 47/48

(52) U.S. Cl. CPC A61K 47/48284
USPC 514/15.2

(57) ABSTRACT

The present invention relates to methods and compositions for treating cancer. Some embodiments include methods comprising increasing expression of a nucleic acid encoding secreted protein acidic and rich in cysteine (SPARC) protein or SPARC protein or the level or activity of SPARC protein and administering a chemotherapeutic agent to a subject in need thereof.

### RELATED APPLICATIONS

This application claims priority to U.S. Provisional Application No. 61/487,202 filed May 17, 2011 entitled “COMBINATION THERAPY FOR TREATMENT OF CANCER” and U.S. Provisional Application No. 61/462,431 filed Feb. 2, 2011 entitled “COMBINATION THERAPY FOR TREATMENT OF CANCER” the disclosures of which are each incorporated herein by reference in their entireties.

### FIELD OF THE INVENTION

The present invention relates to methods and compositions for treating cancer. Some embodiments include methods comprising increasing expression of a nucleic acid encoding secreted protein acidic and rich in cysteine (SPARC) protein or SPARC protein or the level or activity of SPARC protein and administering a chemotherapeutic agent to a subject in need thereof.

### BACKGROUND OF THE INVENTION

Breast cancer is the most common cancer among women (excluding basal and squamous cell skin cancer) in the U.S. and is the second most common cause of cancer death among women. In the year 2007, the estimated new cases of breast cancer among women in the U.S. is 178,480 and the estimated death from breast cancer is 40,460 (1). Currently, there is no cure for patients with metastatic breast cancer. In addition, despite the usefulness of adjuvant chemotherapy and hormonal agents, many patients still relapse and die of recurrent or metastatic breast cancer. Chemotherapy is often used in the treatment of many patients with advanced breast cancer. Doxorubicin, paclitaxel, and docetaxel are some of the most active and commonly used drugs. However, the disease often progresses in a few months, even with combination chemotherapy. In a study comparing paclitaxel, doxorubicin, or the combination of both as first line therapy, Sledge et al (2) reported a median time to treatment failure of 5.8 to 8 months, and overall survival ranging from 18.9 to 22.2 months. Other studies, albeit with a smaller number of patients, reported similar results (3-5).

Since many patients with recurrent or metastatic disease already received doxorubicin as adjuvant treatment at initial diagnosis, taxanes are often the chemotherapy of choice due to their potent activity as single agents. In a phase III trial comparing docetaxel and mitomycin/vinblastine combination in the treatment of patients pre-treated with doxorubicin, Nabholtz et al. (6) showed a median time to disease progression of 4.4 and 2.5 months, respectively, with median overall survival of 11.4 months for docetaxel and 8.7 months for the combination. Other phase III clinical trials comparing docetaxel and paclitaxel, docetaxel methotrexate/5-fluorouracil, or docetaxel and 5-fluorouracil/vinorelbine have demonstrated similar findings, with median time to disease progression of 3 to 6.5 months, and median overall survival of 10.4 to 16 months (7-9). Even in patients with doxorubicin-naive metastatic breast cancer, similar results were reported from several phase III clinical trials (10-12). Therefore, there is an urgent need for better treatments.

### SUMMARY OF THE INVENTION

Some embodiments of the present invention include methods of treating cancer in a subject in need thereof comprising: increasing expression of a nucleic acid encoding secreted protein acidic and rich in cysteine (SPARC) protein or the level or activity of SPARC protein or increasing the expression or activity of SPARC protein in a tumor cell of the subject; and administering an albumin-bound chemotherapeutic agent.

In some embodiments, increasing expression of a nucleic acid encoding secreted protein acidic and rich in cysteine (SPARC) protein or the level or activity of SPARC protein or increasing expression or activity of SPARC protein comprises administering a hypomethlyating agent or a histone deacetylase inhibitor.

In some embodiments, the hypomethlyating agent is selected from the group consisting of azacitidine, and decitabine.

In some embodiments, the inhibitor is selected from the group consisting of vorinostat and valproic acid.

In some embodiments, the albumin-bound chemotherapeutic agent comprises an agent selected from the group consisting of paclitaxel, docetaxel, and rapamycin.

In some embodiments, the albumin-bound chemotherapeutic agent is Abraxane.

In some embodiments, the albumin-bound chemotherapeutic agent is administered weekly.

In some embodiments, the cancer comprises a cancer selected from the group consisting of an advanced solid tumor, a metastatic solid tumor, a lymphoma, ovarian cancer, endometrial cancer, lung cancer, a sarcoma, pancreatic cancer, a leukemia, and breast cancer.

In some embodiments, increasing expression of a nucleic acid encoding secreted protein acidic and rich in cysteine (SPARC) protein or the level or activity of SPARC protein or increasing expression or activity of SPARC protein in a tumor cell of the subject comprises administering at least about 75 mg/m² azacitadine, and administering an albumin-bound chemotherapeutic agent comprises administering at least about 100 mg/m² nab-paclitaxel.

In some embodiments, the nab-paclitaxel is administered weekly.

In some embodiments, the nab-paclitaxel is administered for at least 2 weeks.

In some embodiments, the azacitadine is administered prior to administration of nab-paclitaxel.

In some embodiments, azacitadine is administered daily for an initial period and subsequent to said initial period nab-paclitaxel is administered periodically.

In some embodiments, the initial period is 5 days.

In some embodiments, nab-paclitaxel is administered on days 8, 15, and 22.

In some embodiments, the administration of azacitadine and subsequent administration of nab-paclitaxel is repeated for a plurality of cycles.

In some embodiments, the plurality of cycles is 6 cycles.

### DETAILED DESCRIPTION

The present invention relates to methods and compositions for treating cancer. Some embodiments include methods comprising increasing expression of a nucleic acid encoding secreted protein acidic and rich in cysteine (SPARC) protein or the level or activity of SPARC protein and administering a chemotherapeutic agent to a subject in need thereof. In some embodiments, the chemotherapeutic agent comprises an albumin-associated or albumin-bound chemotherapeutic agent. In some embodiments, increasing expression of a nucleic acid encoding SPARC protein or the level or activity of SPARC protein can include administering a hypomethlyating agent, or a histone deacetylase inhibitor.

It has been discovered that treatment with agents that can enhance the expression of a nucleic acid encoding SPARC protein or the level or activity of SPARC protein can increase the efficacy of chemotherapeutic agents. In some embodiments enhanced expression of SPARC protein in tumor cells can increase the efficacy of albumin-bound or albumin-associated chemotherapeutic agents such as nanoparticle paclitaxel (Abraxane∩). In addition, in some embodiments, agents that enhance expression of SPARC may also suppress tumor growth.

Applicants have discovered that the efficacy of nanoparticle albumin-bound paclitaxel correlates with the expression of tumor-associated SPARC. A common mechanism for SPARC downregulation is hypermethylation. A study described herein investigated the use of the hypomethylating agent azacitadine, followed by paclitaxel in the treatment of refractory advanced solid tumors.

### Secreted Protein Acidic and Rich in Cysteine (SPARC) Protein

SPARC, secreted protein acidic and rich in cysteine, is a secreted glycoprotein that forms a transient component of the extracellular matrix (ECM), and is involved in morphogenesis, tissue remodeling, and cell migration and proliferation through cell-ECM interactions. In addition, SPARC has also been found to bind serum albumin with high affinity (Sage H, Johnson C, Bornstein P. Characterization of a novel serum albumin-binding glycoprotein secreted by endothelial cells in culture. J Biol Chem. 1984. 259(6):3993-4007). Over-expression of SPARC is seen in some tumors. The accumulation of Abraxane at tumor site due to its binding to SPARC protein is thought to be one of the mechanisms why Abraxane, an albumin-bound paclitaxel, works well in some tumors (Gradishar W J. Albumin-bound paclitaxel: a next-generation taxane. Expert Opin Pharmacother. 2006 June; 7(8):1041-53). The expression and function of SPARC seem to be tumor type dependent.

SPARC has been shown to act as a tumor suppressor in many other tumor types (Tai I T, Dai M, Owen D A, et al. Genome-wide expression analysis of therapy-resistant tumors reveals SPARC as a novel target for cancer therapy. J Clin Invest. 2005. 115(6):1492-502; Sato N, Fukushima N, Maehara N, et al. SPARC/osteonectin is a frequent target for aberrant methylation in pancreatic adenocarcinoma and a mediator of tumor-stromal interactions. Oncogene. 2003. 22(32):5021-30; Mok S C, Chan W Y, Wong K K, et al. SPARC, an extracellular matrix protein with tumor-suppressing activity in human ovarian epithelial cells. Oncogene. 1996. 12(9):1895-901; Yiu G K, Chan W Y, Ng S W, et al. SPARC (secreted protein acidic and rich in cysteine) induces apoptosis in ovarian cancer cells. Am J Pathol. 2001. 159(2):609-22; and Said N, Motamed K. Absence of host-secreted protein acidic and rich in cysteine (SPARC) augments peritoneal ovarian carcinomatosis. Am J Pathol. 2005. 167:1739-52).

Using gene-expression microarray, SPARC has been shown to be a putative resistance-reversal gene and reexpression of SPARC conferred radio- and chemosensitivity to resistant colon cancer cells in a xenograft mouse model. SPARC expression was down-regulated or absent in most colon cancer cell lines and primary colon cancers. SPARC promoter hypermethylation was seen in most colon cancer cell lines and in all of primary colon cancer tissues samples. The demethylating agent, 5Aza-dC, was able to upregulate SPARC expression in most cases (Yang E, Kang H J, Koh K H, et al. Frequent inactivation of SPARC by promoter hypermethylation in colon cancers. Int J Cancer. 2007. 121(3):567-75). Similarly, most pancreatic cancer cell lines lacked SPARC expression, and this was associated with hypermethylation of its promoter. A demethylating agent was able to induce SPARC expression in these cell lines. Addition of exogenous SPARC protein suppressed the growth of pancreatic cancer cells.

Loss of SPARC expression and hypermethylation of SPARC promoter were observed in many lung cancer cell lines as well as primary lung cancer tissues (Ito M, Ito G, Kondo M, et al. Frequent inactivation of RASSF1A, BLU, and SEMA3B on 3p21.3 by promoter hypermethylation and allele loss in non-small cell lung cancer. Cancer Lett. 2005. 225(1):131-9; Suzuki M, Hao C, Takahashi T, et al. Aberrant methylation of SPARC in human lung cancers. Br J Cancer. 2005. 92(5):942-8). Reexpression was seen in all cases after treatment with a demethylating agent. Similar findings were also observed with invasive cervical cancer (Soya P, Feng Q, Geiss G, et al. Discovery of novel methylation biomarkers in cervical carcinoma by global demethylation and microarray analysis. Cancer Epidemiol Biomarkers Prey. 2006. 15(1):114-23). Downregulation of SPARC expression was also found in ovarian cancer. Overexpression of SPARC or treatment with SPARC induced apoptosis and suppressed the growth and tumoriginicity of ovarian cancer cells. SPARC-deficient mice were found to have shortened survival and developed extensive intraperitoneal carcinomatosis. For breast cancer, high expression of SPARC mRNA was found to correlate with poor survival in one study (Watkins G, Douglas-Jones A, Bryce R, et al. Increased levels of SPARC (osteonectin) in human breast cancer tissues and its association with clinical outcomes. Prostaglandins Leukot Essent Fatty Acids. 2005. 72(4):267-72), but SPARC protein expression detected by immunohistochemistry, which was positive in 54.2% of 253 cases of infiltrating ductal carcinoma tested, had no correlation with 5-year survival (Kim Y W, Park Y K, Lee J, et al. Expression of osteopontin and osteonectin in breast cancer. J Korean Med Sci. 1998. 13(6):652-7). A recent study showed that endogenous SPARC expression inhibited breast cancer cell metastasis (Koblinski J E, Kaplan-Singer B R, VanOsdol S J, et al. Endogenous osteonectin/SPARC/BM-40 expression inhibits MDA-MB-231 breast cancer cell metastasis. Cancer Res. 2005. 65(16):7370-7).

### Albumin-Bound or Albumin-Associated Agents

Some embodiments of the present invention include albumin-bound or albumin-associated chemotherapeutic agents. Nanoparticle paclitaxel (Abraxane™) is an example of an albumin-bound agent and comprises an albumin-stabilized nanoparticle formulation. Abraxane can increase the intra-tumoral concentration of the associated chemotherapeutic activity.

Without wishing to be bound to any one theory, Abraxane may increase the local concentration of the associated chemotherapeutic activity by a receptor-mediated transport process that includes an albumin-specific receptor (gp60). Activation of gp60 activates caveolin-1. Activation of cavelin-1, in turn, activates the formation of caveolae in the endothelial wall which transport the albumin-bound chemotherapeutic complex to the tumor interstitium (13). A protein specifically secreted by the tumor (SPARC) binds and entraps the albumin, allowing release of a hydrophobic drug to the tumor cell membrane or the release of a chemotherapeutic agent into the cell (14). Abraxane is the first biologically interactive nanoparticle leveraging this gp-60/caveolin-1/caveolae/SPARC pathway to increase intra-tumoral concentration of the drug and reducing toxic drug in normal tissue.

Preclinical Studies with Abraxane

Preclinical studies comparing Abraxane to Taxol demonstrated lower toxicities for Abraxane, with a maximum tolerated dose (MTD) approximately 50% higher for Abraxane compared to Taxol. At equal doses there was less myelosuppression and improved efficacy in a xenograft tumor model of human mammary adenocarcinoma. At equitoxic doses of paclitaxel, Abraxane was found to be markedly more efficacious than Taxol (15).

Clinical Studies with Abraxane

### Every 3 Weeks Schedule

In a phase I study, the maximum tolerated dose of Abraxane was determined to be 300 mg/m² by 30 minute infusion every 3 weeks, without premedication or G-CSF support (16). No severe hypersensitivity reactions occurred with Abraxane despite the absence of premedication. Dose-limiting toxicities included sensory neuropathy, stomatitis, and superficial keratopathy, which occurred at a dose of 375 mg/m².

Two multicenter phase II studies have evaluated 2 dose levels of Abraxane (300 mg/m², n=63, and 175 mg/m², n=43) in patients with metastatic breast cancer (17). The overall response rates in these two phase II trials were 40% (95% CI 25-54%) for the 175 mg/m² dose, and 48% (95% CI 35-60%) for the 300 mg/m² dose. Of 39 patients receiving 300 mg/m² as first-line therapy for metastatic breast cancer, 64% (95% CI 49-79%) responded. This was contrasted with a 45% response rate in similar patients at the lower dose level. Grade 4 neutropenia was noted in 24% of patients at the higher dose level, occurred primarily during the first cycle and resolved rapidly.

A Phase III trial in patients with metastatic breast cancer compared Abraxane 260 mg/m² to Taxol 175 mg/m² given every 3 weeks (18). Efficacy analyses were based on the ITT population. The objective response rate (ORR) was significantly greater for Abraxane than for Taxol for all patients (33% v 19%, respectively; P=0.001), patients who received first-line therapy (42% v 27%, respectively; P=0.029), patients who received second-line or greater therapy (27% v 13%, respectively; P=0.006), and patients who had received prior anthracycline therapy in either the adjuvant/metastatic setting (34% v 18%, respectively; P=0.002) or the metastatic setting only (27% v 14%, respectively; P=0.010). Tumor response rate was also significantly higher for Abraxane than for Taxol in patients with visceral dominant lesions (34% v 19%, respectively; P=0.002) and in patients aged younger than 65 years (34% v 19%, respectively; P<0.001). The objective response rate also was greater for Abraxane compared with standard paclitaxel in patients with nonvisceral dominant lesions (34% v 19%, respectively) and in patients ≧65 years old (27% v 19%, respectively), but the results did not reach statistical significance because of the small number of patients in these subsets.

Median TTP was significantly longer with Abraxane than with Taxol for all patients (23.0 v 16.9 weeks, respectively; hazard ratio [HR]=0.75; P=0.006). There was a trend for greater median survival for all patients treated with Abraxane than with Taxol (65.0 v 55.7 weeks, respectively; P=0.374). Although no difference in survival was observed in first-line patients, the difference was statistically significant in patients who received Abraxane, compared with Taxol, as second-line or greater therapy (56.4 v 46.7 weeks, respectively; HR=0.73; P=0.024) (18).

The incidence of hypersensitivity reactions (any grade) was low for both arms (1% for Abraxane and 2% for Taxol). No severe (grade 3 or 4) treatment-related hypersensitivity reactions occurred in any of the patients in the Abraxane group despite the absence of premedication. In contrast, grade 3 hypersensitivity reactions occurred in the Taxol group despite standard premedication (chest pain, two patients; allergic reaction, three patients). Per protocol, corticosteroids and antihistamines were not administered routinely to patients in the Abraxane group; however, premedication was administered for emesis, myalgia/arthralgia, or anorexia in 18 patients (8%) in the Abraxane group in 2% of the treatment cycles, whereas 224 patients (>99%) in the Taxol group received premedication in 95% of the cycles.

Although the patients in the Abraxane group received an average paclitaxel dose-intensity 49% greater than that received by patients in the Taxol group, the incidence of treatment-related grade 4 neutropenia was significantly lower in the Abraxane group than in the Taxol group (9% v 22%, respectively; P<0.001), with a higher mean neutrophil nadir (1.67 v 1.31×10⁹/L, respectively; P=0.046), suggesting that polyethylated castor oil may have contributed to this toxicity in patients who received standard paclitaxel.

As expected with a higher dose of paclitaxel, treatment-related grade 3 sensory neuropathy occurred more frequently in the Abraxane arm than in the Taxol arm (10% v 2%, respectively; P<0.001); however, these episodes improved with interruption of treatment to grade 2 or 1 in a median 22 days and were easily managed with treatment interruption and dose reduction. By day 28 after its first occurrence, the number of patients with persistent grade 3 sensory neuropathy was the same (n=4) in both study arms. No episodes of motor neuropathy or grade 4 sensory neuropathy were reported in either group.

The only clinical chemistry value that was notably different between the two treatment arms was higher serum glucose levels in the Taxol-treated patients, who also had a higher incidence of hyperglycemia reported as an AE compared with Abraxane-treated patients (7% v 1% respectively; P=0.003).

Subgroup analyses revealed that the safety profiles of Abraxane and Taxol in patients who received the drugs as first-line therapy were similar to those in the overall study population. In subgroup analyses by age, the reported AEs were similar in patients less than 65 years old and patients ≧65 years old in both groups. Of the patients ≧65 years old, the incidences of the following AEs were notably lower in the Abraxane group than in the Taxol group: neutropenia (23% v 59%, respectively), leukopenia (10% v 31%, respectively), nausea (20% v 38%, respectively), hyperglycemia (0% v 19%, respectively), and flushing (0% v 16%, respectively). These data indicate no additional safety concerns for Abraxane in patients ≧65 years old compared with younger patients.

Six patients (3%) in the Abraxane group and eight patients (4%) in the standard paclitaxel group died during the study, all as a result of disease progression. No treatment-related deaths occurred in the Abraxane group; one patient (<1%) in the Taxol group died of multiorgan failure, which was considered by the investigator to be possibly related to treatment but may also have been a result of sepsis and/or progressive disease.

### Weekly for 3 Weeks, Every 4 Weeks Schedule

Thirty-nine patients were enrolled into A Phase I study of Abraxane administered weekly for 3 weeks followed by a 1 week rest in patients with advanced solid tumors (19). The maximum tolerated doses for heavily and lightly pre-treated patients were 100 and 150 mg/m² respectively. Dose limiting toxicities included grade 4 neutropenia and grade 3 sensory neuropathy. Premedication was not required, and unexpected, non-taxane associated toxicities were not observed.

In a Phase II trial in heavily pretreated patients with taxane-refractory metastatic breast cancer, objective antitumor responses occurred in 15% of women treated with Abraxane 100 mg/m² on this schedule (20). Abraxane weekly regimen was well tolerated. 91% of patients were treated at the full dose of 100 mg/m2 of Abraxane without dose reductions. Based on the activity and low toxicity documented with the Abraxane 100 mg/m² weekly regimen, this study was expanded to evaluate the efficacy and safety/tolerability of a higher dose of Abraxane 125 mg/m² weekly regimen in 75 additional patients. Results of this dose-finding study confirm the dose of Abraxane 100 mg/m² as the appropriate dose for further study in this patient population (21).

### Weekly Schedule

The administration of Abraxane in a neoadjuvant setting to patients with locally advanced breast cancer at a dose of 100 mg/m² weekly for 12 weeks, with no break was studied (22). Four cycles of FEC were administered sequentially based on patients' HER2 status: HER2 negative patients received FEC-100 (F: 500 mg/m², E: 100 mg/m², C: 500 mg/m² Q3 weeks) and HER2 positive patients received weekly trastuzumab in addition to FEC-75 (F: 500 mg/m², E: 75 mg/m², C: 500 mg/m² Q3 weeks). Weekly trastuzumab was permitted during Abraxane and FEC-75 treatment at the discretion of the investigator. The primary objective of the trial was to determine the pathologic complete response rate in the breast. At the time of initial report, 65 patients had been entered on study and were evaluable for clinical complete response rate and safety. Following 12 weeks of Abraxane, a clinical complete response rate of 32% was noted. The therapy was well tolerated, with 48/65 patients receiving 12 doses in 12 weeks and 13/65 receiving 12 doses in 13-14 weeks. The incidence of peripheral (sensory) neuropathy was low (11% grade 2 and 5% grade 3) as was neutropenia (3% grade 3 and no grade 4). The authors concluded that the administration of Abraxane 100 mg/m² weekly×12 was both effective and tolerable.

### Azacitadine (Vidaza™)

Azacitidine, an analog of the pyrimidine nucleoside cytidine, has effects on cell differentiation, gene expression, and DNA synthesis and metabolism. Since the early 1970s, azacitidine has been investigated primarily in the US for the treatment of acute leukemia. Clinical studies have focused mainly on patients with disease refractory to conventional chemotherapy. Results of these investigations demonstrated activity of azacitidine in the treatment of AML. Clinical studies subsequently evaluated the effects of azacitidine in a variety of other malignant and hematologic disorders, including solid tumors, hemoglobinopathies (eg, thalassemia and sickle cell anemia), and MDS. In 1984, the Cancer and Leukemia Group B (CALGB) began a series of clinical studies with azacitidine in patients with MDS. These studies, in addition to other supportive data, led to the approval of Vidaza® (azacitidine) in May 2004 for the treatment of MDS.

Azacitidine inhibits the methylation of newly synthesized DNA by inhibiting DNA methyltransferase (DNMT).⁵⁰⁻⁵² Increased methylation of DNA (hypermethylation) may result in the silencing of critical genes responsible for cell growth control and differentiation. Hypermethylation of CpG islands spanning the promoter regions of tumor suppressor genes is commonly associated with cancers.⁵³ It is now widely recognized that hypermethylation of DNA is frequently associated with myelodysplastic syndromes and other cancers,⁵⁴⁻⁵⁶ such as renal,⁵⁷ melanoma,⁵⁸ breast,⁵⁹ colorectal,⁶⁰ non-small cell lung⁶¹ and hematologic malignancies.⁶² Azacitidine is believed to exert its antineoplastic effects through hypomethylation and cytotoxicity on abnormal hematopoietic cells in the bone marrow.⁶³⁻⁶⁷ Hypomethylation may restore normal function to genes that are critical for differentiation and proliferation.^{53, 68, 69} The cytotoxic effects of azacitidine cause the death of rapidly dividing cells, including cancer cells that are no longer responsive to normal growth control mechanisms.^{63, 70-72}

The cytotoxicity of azacitidine is proportional to dose and exposure time.^{63, 64} Although the mechanisms of cytotoxicity are complex and multifaceted, there is general agreement that incorporation of azacitidine into DNA and RNA, and inhibition of protein synthesis, are critically important.⁷³ Cytotoxicity is greatest in cells that are proliferating (S phase) and metabolically active.⁶³ Cytotoxic effects may also be mediated through induction of the DNA damage response pathways.⁷² Nonproliferating cells are relatively insensitive to azacitidine.⁶³

Preclinical and Pharmacokenetic Studies with Azacitadine

Toxicology studies have been conducted in mice, rats, dogs, and Rhesus monkeys.⁷⁴ Most of the studies were performed during the 1970s and early 1980s according to existing guidelines and standards in place during that period. The preclinical studies identified the bone marrow, liver, kidneys, and lymphoid tissues (spleen, lymph nodes, and thymus) as the main target organs of toxicity for azacitidine.⁷⁴ In single-dose studies, the lethal dose of azacitidine after intravenous (IV) administration in mice, rats, and dogs was approximately 250 mg/m². Repeated daily dosing appears to increase the toxicity of azacitidine.⁷⁴ The genotoxicity of azacitidine is consistent with that of other nucleoside analogs that interact with nucleic acids.⁷⁴ Likewise, similar to other agents with cytostatic properties, azacitidine was embryotoxic and reduced the reproductive performance in mice and rats.⁷⁴

Limited azacitidine pharmacokinetic data are currently available. Based on human plasma concentrations of total radioactivity (which represents parent drug plus circulating metabolites), azacitidine is rapidly absorbed when given subcutaneously (SC), with maximum plasma concentrations found 0.5 to 2 hours after dosing.⁷⁴ Azacitidine and/or its by-products are then rapidly cleared by the kidneys. The half-lives and percent radioactivity recovered in urine are similar following IV and SC routes of administration. The effects of renal or hepatic impairment, gender, age, or race on the pharmacokinetics of azacitidine have not been studied.⁷⁴ A single dose (75 mg/m²) SC versus IV crossover study in 6 MDS subjects⁷⁵ revealed an approximate bioavailability of 89% for the SC dose (range 52% to 128%) with mean half-lives of 0.69 hour and 0.36 hour after SC and IV administration, respectively. These results demonstrated that azacitidine is rapidly and nearly completely absorbed after SC administration and that elimination is also rapid. The apparent SC clearance (167 L/h or 2791 mL/min) and systemic IV clearance (147 L/h) of azacitidine exceeded the glomerular filtration rate (approximately 125 mL/min) and total renal blood flow (1200 mL/min) in healthy subjects. This indicates that non-renal elimination (eg, metabolism, hydrolysis, and/or degradation) plays a role in the elimination of parent azacitidine. In addition, azacitidine 75 mg/m² was generally well-tolerated after single SC injection or IV infusion over 10 minutes.⁷⁵

A number of studies have looked at different parenteral doses and schedules of azacitidine, finding maximum tolerated doses of up to 500 mg/m² when administered weekly.⁷⁶

Clinical Studies with Azacitadine

During the two decades between the start of the CALGB studies and the approval of azacitidine, a new understanding of MDS has developed, such as the World Health Organization (WHO) diagnostic criteria, the International Prognostic Scoring System (IPSS), and the International Working Group (IWG) response criteria. Silverman et al. reevaluated the combined data (N=309) from 3 of the CALGB studies using the WHO classification system for MDS and AML and the IWG response criteria.⁷⁷ Using the IWG response criteria in MDS patients, response rates were between 40% and 70% in azacitidine treated patients. Ten to 17% of patients achieved a complete remission; partial remission was rare; and 23% to 36% of patients had a hematologic improvement. In patients with AML (N=103), 35% to 48% had hematologic improvement or better responses. The median survival time for 27 patients assigned to azacitidine was 19.3 months compared with 12.9 months for the 25 patients assigned to observation.⁷⁷

A randomized international Phase III trial (Study AZA PH GL 2003 CL 001) for higher-risk MDS patients, classified by FAB as RAEB, RAEB-T, or CMML with 0-29% marrow blasts, with an IPSS of Intermediate −2 or High by central pathology/cytogenetic review was recently reported.⁷⁸ Patients were randomized to azacitidine (75 mg/m²/day×7days in 28 day cycles) or conventional care regimens, where a conventional care regimen was pre-selected by the Investigator as best supportive care (transfusions, antibiotics, and G-CSF for neutropenic infection), low-dose cytarabine (20 mg/m²/day×14 days in 28 day cycles); or standard chemotherapy (conventional induction/consolidation). Patients were stratified by FAB/IPSS and randomized 1:1 to azacitidine or a conventional care regimen. This trial did not allow erythropoietin. Three-hundred fifty eight patients (70% male) were randomized at 79 centers to azacitidine (N=179) or a conventional care regimen (N=179): best supportive care only (N=105, 59%), low-dose cytarabine (N=49, 27%), or standard chemotherapy (N=25, 14%). Median age was 69 years (range 38-88 years). The azacitidine and conventional care regimen groups were comparable for baseline patient characteristics. At baseline, 95% of patients were higher risk: RAEB (58%), RAEB-T/WHO AML (34%), CMML (3%), and other (5%). By IPSS, 87% were higher risk: Intermediate −2 (40%), High (47%), and 13% Indeterminate/other. Azacitidine was administered for a median of 9 cycles; low-dose cytarabine for 4 cycles. Median follow-up for the survival analysis was 21.1 months. Azacitidine demonstrated statistically superior overall survival compared to a conventional care regimen, with a median overall survival of 24.4 months vs. 15 months for a conventional care regimen (stratified log-rank p=0.0001, hazard ration 0.58). Two-year survival approximately doubled in the azacitidine arm compared to a conventional care regimen: 51% vs. 26% (p<0.0001). Azacitidine was well tolerated with safety data consistent with previous reports.

Further details can be found in the azacitidine Investigator's Brochure, which contains comprehensive pharmacology, toxicology, pharmacokinetics, pharmacodynamics, metabolism, preclinical, and clinical efficacy and safety data information.⁷⁴

### SPARC Protein and Cancer

SPARC, secreted protein acidic and rich in cysteine, is a secreted glycoprotein that forms a transient component of the extracellular matrix (ECM), and is involved in morphogenesis, tissue remodeling, and cell migration and proliferation through cell-ECM interactions (25-27). SPARC has also been found to interact with other components of the ECM and to regulate the expression and function of matrix metalloproteinase (25-27).

In addition, SPARC has also been found to bind serum albumin with high affinity (28). Over-expression of SPARC is seen in some tumors. The accumulation of Abraxane at tumor site due to its binding to SPARC protein is thought to be one of the mechanisms how Abraxane, an albumin-bound paclitaxel, works well in some tumors (Review in Ref. 29). The expression and function of SPARC seem to be tumor type dependent. In melanoma, SPARC over-expression is seen in the majority of primary and metastatic melanoma and seems to be associated with tumorigenicity and progression of the disease (30, 31). Similarly, SPARC expression in glioma is associated with invasion and progression (32-34).

In contrast, SPARC has been shown to act as a tumor suppressor in many other tumor types (35-39). The underlying mechanisms for this function are not currently clear. However, SPARC was shown to act as an extracellular modulator of Ca⁺² and other ECM proteins, and was associated with changes in cell shape and inhibition of cell spreading (40). In addition, recent studies have shown that SPARC inhibited the proliferation and migration of endothelial cells, thereby inhibiting angiogenesis (41), enhanced tumor stroma formation and impaired fibroblast activation, promoting a non-permissible tumor microenvironment (42).

Using gene-expression microarray, SPARC has been shown to be a putative resistance-reversal gene and reexpression of SPARC conferred radio- and chemosensitivity to resistant colon cancer cells in a xenograft mouse model (35). SPARC expression was down-regulated or absent in most colon cancer cell lines and primary colon cancers. SPARC promoter hypermethylation was seen in most colon cancer cell lines and in all of primary colon cancer tissues samples. The demethylating agent, 5Aza-dC, was able to upregulate SPARC expression in most cases (43). Similarly, most pancreatic cancer cell lines lacked SPARC expression, and this was associated with hypermethylation of its promoter. A demethylating agent was able to induce SPARC expression in these cell lines. Addition of exogenous SPARC protein suppressed the growth of pancreatic cancer cells (36).

Loss of SPARC expression and hypermethylation of SPARC promoter were observed in many lung cancer cell lines as well as primary lung cancer tissues (44, 45). Reexpression was seen in all cases after treatment with a demethylating agent (45). Similar findings were also observed with invasive cervical cancer (46). Downregulation of SPARC expression was also found in ovarian cancer (37). Overexpression of SPARC (37) or treatment with SPARC (38) induced apoptosis and suppressed the growth and tumoriginicity of ovarian cancer cells. SPARC-deficient mice were found to have shortened survival and developed extensive intraperitoneal carcinomatosis (39). For breast cancer, high expression of SPARC mRNA was found to correlate with poor survival in one study (47), but SPARC protein expression detected by immunohistochemistry, which was positive in 54.2% of 253 cases of infiltrating ductal carcinoma tested, had no correlation with 5-year survival (48). A study showed that endogenous SPARC expression inhibited breast cancer cell metastasis (49).

### Method of Treating Cancer

Some embodiments include methods of treating cancer in a subject in need thereof. Some such embodiments include increasing expression of a nucleic acid encoding secreted protein acidic and rich in cysteine (SPARC) protein or the level or activity of SPARC protein or increasing expression of SPARC protein or activity of SPARC protein in a tumor cell of the subject, and administering an albumin-bound or albumin-associated chemotherapeutic agent.

In some embodiments, increasing expression of a nucleic acid encoding secreted protein acidic and rich in cysteine (SPARC) protein or the level or activity of SPARC protein or increasing expression or activity of SPARC protein comprises administering a hypomethlyating agent or a histone deacetylase inhibitor. Examples of hypomethlyating agents include azacitidine, and decitabine. Examples of histone deacetylase inhibitors include vorinostat and valproic acid.

In some embodiments, the nanoparticle albumin-bound chemotherapeutic agent comprises an agent selected from the group consisting of paclitaxel, docetaxel, rapamycin.

In some embodiments, the nanoparticle albumin-bound chemotherapeutic agent is Abraxane.

Some embodiments include administering an albumin-bound or albumin-associated chemotherapeutic agent, such as Abraxane, in the range of between about 10 mg/m² and 200 mg/m², between about 20 mg/m² and 180 mg/m², between about 30 mg/m² and 170 mg/m², between about 40 mg/m² and 160 mg/m², between about 50 mg/m² and 150 mg/m², between about 60 mg/m² and 140 mg/m², between about 70 mg/m² and 120 mg/m², between about 80 mg/m² and 110 mg/m², and between about 90 mg/m² and 100 mg/m². Some embodiments include administering at least about 10 mg/m², at least about 20 mg/m², at least about 25 mg/m², at least about 30 mg/m², at least about 35 mg/m², at least about 40 mg/m², at least about 45 mg/m², at least about 50 mg/m², at least about 55 mg/m², at least about 60 mg/m², at least about 65 mg/m², at least about 70 mg/m², at least about 75 mg/m², at least about 80 mg/m², at least about 85 mg/m², at least about 90 mg/m², at least about 95 mg/m², at least about 100 mg/m², at least about 105 mg/m², at least about 110 mg/m², at least about 115 mg/m², at least about 120 mg/m², at least about 125 mg/m², at least about 130 mg/m², at least about 135 mg/m², at least about 140 mg/m², at least about 145 mg/m², at least about 150 mg/m², at least about 155 mg /m², at least about 160 mg/m², at least about 165 mg/m², at least about 170 mg/m², at least about 175 mg/m², at least about 180 mg/m², at least about 185 mg/m², at least about 190 mg/m², at least about 195 mg/m², and at least about 200 mg/m² of an albumin-bound or albumin-associated chemotherapeutic agent, such as Abraxane.

In some embodiments, the nanoparticle albumin-bound chemotherapeutic agent is administered at least about twice daily, once daily, at least once weekly, weekly, at least once every two weeks, at least once monthly, or monthly. In some embodiments, the nanoparticle albumin-bound chemotherapeutic agent is administered at least once every 1 day, at least once every 2 days, at least once every 3 days, at least once every 4 days, at least once every 5 days, at least once every 6 days, or at least once every 7 days. In some embodiments, the nanoparticle albumin-bound chemotherapeutic agent is administered at least once every 1 week, at least once every 2 weeks, at least once every 3 weeks, or at least once every 4 weeks.

In some embodiments, increasing expression of a nucleic acid encoding secreted protein acidic and rich in cysteine (SPARC) protein or the level or activity of SPARC protein or increasing expression or activity of SPARC protein comprises administering a hypomethlyating agent or a histone deacetylase inhibitor, such as azacitadine, in the range of between about 10 mg/m² and 150 mg/m², between about 20 mg/m² and 140 mg/m², between about 30 mg/m² and 130 mg/m², between about 40 mg/m² and 120 mg/m², between about 50 mg/m² and 100 mg/m², between about 60 mg/m² and 90 mg/m², and between about 70 mg/m² and 80 mg/m². Some embodiments include administering at least about 10 mg/m², at least about 20 mg/m², at least about 25 mg/m², at least about 30 mg /m², at least about 35 mg/m², at least about 40 mg/m², at least about 45 mg/m², at least about 50 mg/m², at least about 55 mg/m², at least about 60 mg/m², at least about 65 mg/m², at least about 70 mg/m², at least about 75 mg/m², at least about 80 mg/m², at least about 85 mg/m², at least about 90 mg/m², at least about 95 mg/m², and at least about 100 mg/m², of a hypomethlyating agent or a histone deacetylase inhibitor, such as azacitadine.

In some embodiments, the hypomethlyating agent or a histone deacetylase inhibitor is administered at least about twice daily, once daily, at least once weekly, weekly, at least once every two weeks, at least once monthly, or monthly. In some embodiments, the hypomethlyating agent or a histone deacetylase inhibitor is administered at least once every 1 day, at least once every 2 days, at least once every 3 days, at least once every 4 days, at least once every 5 days, at least once every 6 days, or at least once every 7 days. In some embodiments, the hypomethlyating agent or a histone deacetylase inhibitor is administered at least once every 1 week, at least once every 2 weeks, at least once every 3 weeks, or at least once every 4 weeks.

In some embodiments, the cancer comprises a cancer selected from the group consisting of an advanced solid tumor, a metastatic solid tumor, a lymphoma, ovarian cancer, endometrial cancer, lung cancer, a sarcoma, pancreatic cancer, and breast cancer.

In some embodiments, increasing expression of a nucleic acid encoding secreted protein acidic and rich in cysteine (SPARC) protein or the level or activity of SPARC protein or increasing expression of SPARC protein in a tumor cell of the subject comprises administering at least about 75 mg/m² azacitadine, and administering a nanoparticle albumin-bound chemotherapeutic agent comprises administering at least about 100 mg/m² nab-paclitaxel. In some such embodiments, the nab-paclitaxel is administered weekly. In more such embodiments, the nab-paclitaxel is administered for at least 2 weeks, at least 3 weeks, at least 4 weeks, at least 5 weeks, at least 6 weeks, or more.

### EXAMPLES

### Example 1

### Phase I Study With Azacitadine and Nanoparticle Albumin-Bound Paclitaxel

A Phase I study of the hypomethylating agent, azacitadine, with the nanoparticle albumin-bound- (nab-) paclitaxel in the treatment of patients with advanced or metastatic solid tumors was carried out. The study investigated the use of the hypomethylating agent azacitadine, followed by paclitaxel in the treatment of refractory advanced solid tumors.

### Methods:

Eligible patients include those with solid tumors that progressed or were stable as best response on at least one previous therapy. Enrolled patients include those with metastatic solid tumors who had failed at least one standard treatment. Further criteria included: female and male patients; age≧19 years; ECOG PS≦2; adequate bone marrow, liver and heart function; and signed written-informed consent. Sixteen patients were enrolled in the study and are summarized in Table 1.

TABLE 1

| Characteristic | | |
|---|---|---|
| Number of patients | | 16 |
| Age (y) | Median | 62.5 |
| | Range | 21-83 |
| Gender | Male | 3 |
| | Female | 13 |
| Race | Caucasian | 11 |
| | African American | 4 |
| | Hispanic | 1 |
| Number of prior chemotherapies | 1 | 7 |
| | 2 | 6 |
| | >3 | 3 |
| Tumor type (n = 16) | Ovarian | 6 |
| | Lymphoma | 2 |
| | Lung | 2 |
| | Uterine | 1 |
| | Pancreatic | 1 |
| | Biliary tract | 1 |
| | Sarcoma | 1 |
| | Breast | 1 |
| | Bladder | 1 |

A single center, prospective open study was carried out in which the highest tolerable dose of azacitadine and nab-paclitaxel in patients with advanced or metastatic solid tumors with at least one prior chemotherapy treatment was assessed.

Three dose levels of azacitadine (dose level −1: 50 mg/m², dose level 2: 75 mg/m², or dose level 3: 100 mg/m²) with fixed dose of nab-paclitaxel (100 mg/m² weekly) were evaluated. For each cycle, azacitadine was administered daily for 5 days (Days 1-5), and nab-paclitaxel was administered on Days 8, 15 and 22 on a 28-day cycle, for a total of 6 cycles. Serum and/or tissue samples, where appropriate, were collected for correlative studies.

Outcome evaluation for primary endpoints included: type, incidence, severity, timing, seriousness, and relatedness of adverse events, and laboratory abnormalities; and objective response rate. Outcome evaluation for secondary endpoints included: progression-free survival (PFS); and expression of tissue SPARC protein. Statistical Analysis included the standard 3+3 design. Patients were accrued to each dose level in cohorts of up to 3-6. Escalation was continued until a dose limiting toxicity (DLT) was observed or the highest dose-level was reached. The best response, including complete response (CR), partial response (PR), stable disease (SD), or progression of disease (PD), for each patient was determined. Descriptive statistics were used to summarize all patient characteristics, treatment administration and compliance, and protein biomarkers. Safety data were determined for all patients receiving at least 1 dose of study treatment.

### Results

Patients were initially permitted to have an unlimited number of prior chemotherapies, but after 2 of the first 5 patients experiencing DLT (grade 4 neutropenia >8 days), and dose reduction of nab-paclitaxel occurring in 3 patients all with 4 or more prior cytotoxic regimens, the protocol was amended to permit no more than 2 prior cytotoxic regimens. Cohort 2 was treated at the same dose level (azacitadine 75 mg/m²) with no DLT.

Cohort 3 was treated at the next dose level (azacitadine 100 mg/m²). Two of 4 had DLT of prolonged grade 4 neutropenia. Therefore, the maximum tolerated dose of azacitadine in this regimen is 75 mg/m². Three additional patients were treated at the maximum tolerated dose with no grade 4 toxicity in cycle 1. Two patients were removed before completing cycle 1 because of disease progression. One patient was removed after cycle 4 for noncompliance. Clinical activity included 1 CR in refractory diffuse large B cell (DLBC) lymphoma, 2 CR in ovarian cancer, 4 PR in ovarian and endometrial cancer, 4 SD in lung, sarcoma and pancreatic cancer, 1 unconfirmed PR in breast cancer, and 1 PD in CLL/SLL. Two breast cancer patients in the phase II part had unconfirmed PR and are still receiving treatments. Responses in ovarian cancer patients were calculated based on CA-125 levels. Of 13 accessible patients: 1 CR (7.7%), 7 PR (53.8%), 4 SD (30.8%), for an overall response rate of 61.5%, and disease control rate of 92.3%. Table 2 summarizes the results.

TABLE 2

| Patient | Tumor type | Best response |
|---|---|---|
| 1 | DLBC NHL | CR |
| 2 | Ovarian | PR |
| 3 | Ovarian | CR |
| 4 | Ovarian | N/A |
| 5 | Uterine | PR |
| 6 | NSCLC | SD |
| 7 | Ovarian | PR |
| 8 | Ovarian | PR |
| 9 | Pancreatic | SD |
| 10 | Biliary tract | N/A |
| 11 | Sarcoma | SD |
| 12 | CLL/SLL | PD |
| 13 | Breast | PR |
| 14 | NSCLC | SD |
| 15 | Bladder | N/A |
| 16 | Ovarian | CR |

Conclusions: Azacitadine 75 mg/m² daily for 5 days, followed by weekly nab-paclitaxel 100 mg/m² was well tolerated and seemed to result in dramatic responses in heavily pre-treated patients with cancer of diverse histology.

### Example 2

### Phase II Study Treating Breast Cancer Patients With Azacitadine and Nanoparticle Albumin-Bound Paclitaxel

A Phase II study treating breast cancer patients with azacitadine and nanoparticle albumin-bound- (nab-) paclitaxel is carried out. This study further assesses the safety of the drug combination, and obtains preliminary data on the clinical efficacy of the combination.

### Methods:

Eligible patients include those with pathologically confirmed (Her-2 negative) breast cancer, measurable disease, no prior chemotherapy for metastatic disease. Further criteria include: age≧19 years; ECOG PS≦2; adequate bone marrow, liver and heart function; and signed written-informed consent. Approximately 45 patients are enrolled.

This is a single center, prospective open study. Azacitadine 75 mg/m² followed by weekly nab-paclitaxel 100 mg/m² is administered to patients. For each cycle, azacitadine is administered daily for 5 days (Days 1-5), and nab-paclitaxel is administered on Days 8, 15 and 22 on a 28-day cycle, for a total of 6 cycles. Serum and/or tissue samples, where appropriate, are collected for correlative studies.

Outcome evaluation for primary endpoints include: type, incidence, severity, timing, seriousness, relatedness of adverse events, laboratory abnormalities, objective response rate. Outcome evaluation for secondary endpoints include: progression-free survival (PFS), and expression of tissue SPARC protein. The best response, including complete response (CR), partial response (PR), stable disease (SD), or progression of disease (PD), for each patient is determined summarized. Descriptive statistics are used to summarize all patient characteristics, treatment administration and compliance, and protein biomarkers. Safety data are determined for all patients receiving at least 1 dose of study treatment.

### Results

Patients treated with have a clinical activity including complete response (CR), partial response (PR), stable disease (SD).

Each of the following references is expressly incorporated herein by reference in its entirety and is hereby made a part of this specification.

### REFERENCES

1. American Cancer Society: Cancer Facts and Figures 2007. Atlanta: American Cancer Society; 2007.
2. Sledge G W, Neuberg D, Bernardo P, et al. 2003. Phase III trial of doxorubicin, paclitaxel, and the combination of doxorubicin and paclitaxel as front-line chemotherapy for metastatic breast cancer: an intergroup trial (E1193). J Clin Oncol. 21(4):588-92.
3. Cresta S, Grasselli G, Mansutti M, et al. 2004. A randomized phase II study of combination, alternating and sequential regimens of doxorubicin and docetaxel as first-line chemotherapy for women with metastatic breast cancer. Ann Oncol. 15(3):433-9.
4. Conte P F, Guarneri V, Bruzzi P, et al. 2004. Concomitant versus sequential administration of epirubicin and paclitaxel as first-line therapy in metastatic breast carcinoma: results for the Gruppo Oncologico Nord Ovest randomized trial. Cancer. 101(4):704-12.
5. Alba E, Martin M, Ramos M, et al. 2004. Multicenter randomized trial comparing sequential with concomitant administration of doxorubicin and docetaxel as first-line treatment of metastatic breast cancer: a Spanish Breast Cancer Research Group (GEICAM-9903) phase III study. J Clin Oncol. 22(13):2587-93.
6. Nabholtz J M, Senn H J, Bezwoda W R, et al. 1999. Prospective randomized trial of docetaxel versus mitomycin plus vinblastine in patients with metastatic breast cancer progressing despite previous anthracycline-containing chemotherapy. 304 Study Group. J Clin Oncol. 17(5):1413-24.
7. Jones S E, Erban J, Overmoyer B, et al. 2005. Randomized phase III study of docetaxel compared with paclitaxel in metastatic breast cancer. J Clin Oncol. 23(24):5542-51.
8. Sjostrom J, Blomqvist C, Mouridsen H, et al. 1999. Docetaxel compared with sequential methotrexate and 5-fluorouracil in patients with advanced breast cancer after anthracycline failure: a randomised phase III study with crossover on progression by the Scandinavian Breast Group. Eur J Cancer. 35(8):1194-201.
9. Bonneterre J, Roche H, Monnier A, et al. 2002. Docetaxel vs 5-fluorouracil plus vinorelbine in metastatic breast cancer after anthracycline therapy failure. Br J Cancer. 87(11):1210-5.
10. Bishop J F, Dewar J, Toner G C, et al. 1999. Initial paclitaxel improves outcome compared with CMFP combination chemotherapy as front-line therapy in untreated metastatic breast cancer. J Clin Oncol. 17(8):2355-64.
11. Chan S, Friedrichs K, Noel D, et al. 1999. Prospective randomized trial of docetaxel versus doxorubicin in patients with metastatic breast cancer. J Clin Oncol. 17(8):2341-54.
12. Paridaens R, Biganzoli L, Bruning P, et al. 2000. Paclitaxel versus doxorubicin as first-line single-agent chemotherapy for metastatic breast cancer: a European Organization for Research and Treatment of Cancer Randomized Study with cross-over. J Clin Oncol. 18(4):724-33.
13. Desai N, Trieu V, Yao R, Frankel T, Soon-Shiong P: SPARC expression in breast tumors may correlate to increased tumor distribution of nanoparticle albumin-bound paclitaxel (ABI-007) vs taxol. 2004 SABCS. Abstract No. 206.
14. Desai N, Trieu V, Yao R, Labao E, Soon-Shiong P: Increased endothelial transcytosis of nanoparticle albumin-bound paclitaxel (ABI-007) by gp60-receptors: a pathway inhibited by taxol. 2004 SABCS. Abstract No. 1071.
15. Desai N, Trieu V, Yao Z, et al: Increased Antitumor Activity, Intratumor Paclitaxel Concentrations and Endothelial Cell Transport of Cremophor-Free, Albumin-Bound Paclitaxel, ABI-007, Compared with Cremophor-Based Paclitaxel. Clin Cancer Res. 2006; 12(4). 1317-1324.
16. Ibrahim N K, Desai N, Legha S, et al: Phase I and Pharmacokinetic Study of ABI-007, a Cremophor-free, Protein-stabilized, Nanoparticle Formulation of Paclitaxel. Clin Cancer Research. 2002 May; 8: 1038-1044
17. Ibrahim N K, Samuels B, Page R, et al: Multicenter Phase II Trial of ABI-007, an Albumin-Bound Paclitaxel, in Women with Metastatic Breast Cancer. J Clin Oncol 23:6019-6026, 2005
18. Gradishar W J, Tjulandin S, Davidson N, Shaw H, Desai N, Bhar P, Hawkins M, O'Shaughnessy J. Phase III trial of nanoparticle albumin-bound paclitaxel compared with polyethylated castor oil-based paclitaxel in women with breast cancer. J Clin Oncol. 2005 23(31):7794-803
19. Nyman D W, Campbell K J, Hersh, E, et al: Phase I and Pharmacokinetics Trial of ABI-007, a Novel Nanoparticle Formulation of Paclitaxel in Patients with Advanced Nonhematologic Malignancies. J Clin Oncol 23:7785-7793, 2005
20. Blum J L, et al: Long-term Disease Control in Taxane-Refractory Metastatic Breast Cancer Treated with nab paclitaxel. 2004 ASCO Annual Meeting Proceedings (Post-Meeting Edition). Vol 22, No 14S (July 15 Supplement), 2004: Abstract No. 543.
21. OShaughnessy J A, Blum J L, Sandbach J F, et al: Weekly Nanoparticle Albumin Paclitaxel (Abraxane) Results in Long-Term Disease Control in Patients with Taxane-Refractory Metastatic Breast Cancer. 2004 SABCS. Abstract No. 1070
22. Robidoux A, Buyse M, Buzdar A, et al. Neoadjuvant chemotherapy with sequential weekly nanoparticle albumin-bound paclitaxel (ABI-007, Abraxane® followed by 5-fluorouracil, epirubicin and cyclophosphamide (FEC) in locally advanced breast cancer (LABC): a phase II trial of the NSABP Foundation research programs (FRP) [poster]. Presented at: San Antonio Breast Cancer Symposium; Dec. 14-17, 2006; San Antonio, Tex. Abs 3068.
23. Sullivan M, Hahn K, Kolesar J M. Azacitidine: a novel agent for myelodysplastic syndromes. Am J Health Syst Pharm. 2005 Aug. 1; 62(15):1567-73
24. Kaminskas E, Farrell A T, Wang Y C, Sridhara R, Pazdur R. FDA drug approval summary: azacitidine (5-azacytidine, Vidaza) for injectable suspension. Oncologist. 2005 March; 10(3):176-82
25. Lane T F, Sage E H. The biology of SPARC, a protein that modulates cell-matrix interactions. FASEB J. 1994 February; 8(2):163-73. PMID: 8119487
26. Sage E H, Bornstein P. Extracellular proteins that modulate cell-matrix interactions. SPARC, tenascin, and thrombospondin. J Biol Chem. 1991 Aug. 15; 266(23):14831-4
27. Tremble P M, Lane T F, Sage E H, Werb Z. SPARC, a secreted protein associated with morphogenesis and tissue remodeling, induces expression of metalloproteinases in fibroblasts through a novel extracellular matrix-dependent pathway. J Cell Biol. 1993 June; 121(6):1433-44. PMID: 8509459
28. Sage H, Johnson C, Bornstein P. Characterization of a novel serum albumin-binding glycoprotein secreted by endothelial cells in culture. J Biol Chem. 1984 Mar. 25; 259(6):3993-4007. PMID: 6368555
29. Gradishar W J. Albumin-bound paclitaxel: a next-generation taxane. Expert Opin Pharmacother. 2006 June; 7(8):1041-53. Review. PMID: 16722814
30. Ledda M F, Adris S, Bravo A I, Kairiyama C, Bover L, Chernajovsky Y, Mordoh J, Podhajcer O L. Suppression of SPARC expression by antisense RNA abrogates the tumorigenicity of human melanoma cells. Nat Med. 1997 February; 3(2):171-6.
31. Ledda F, Bravo A I, Adris S, Bover L, Mordoh J, Podhajcer O L. The expression of the secreted protein acidic and rich in cysteine (SPARC) is associated with the neoplastic progression of human melanoma. J Invest Dermatol. 1997 February; 108(2):210-4.
32. Schultz C, Lemke N, Ge S, Golembieski W A, Rempel S A. Secreted protein acidic and rich in cysteine promotes glioma invasion and delays tumor growth in vivo. Cancer Res. 2002 Nov. 1; 62(21):6270-7.
33. Rich J N, Hans C, Jones B, Iversen E S, McLendon R E, Rasheed B K, Dobra A, Dressman H K, Bigner D D, Nevins J R, West M. Gene expression profiling and genetic markers in glioblastoma survival. Cancer Res. 2005 May 15; 65(10):4051-8
34. Shi Q, Bao S, Song L, Wu Q, Bigner D D, Hjelmeland A B, Rich J N. Targeting SPARC expression decreases glioma cellular survival and invasion associated with reduced activities of FAK and ILK kinases. Oncogene. 2007 Jun. 14; 26(28):4084-94
35. Tai I T, Dai M, Owen D A, Chen L B. Genome-wide expression analysis of therapy-resistant tumors reveals SPARC as a novel target for cancer therapy. J Clin Invest. 2005 June; 115(6):1492-502. PMID: 15902309
36. Sato N, Fukushima N, Maehara N, Matsubayashi H, Koopmann J, Su G H, Hruban R H, Goggins M. SPARC/osteonectin is a frequent target for aberrant methylation in pancreatic adenocarcinoma and a mediator of tumor-stromal interactions. Oncogene. 2003 Aug. 7; 22(32):5021-30. PMID: 12902985
37. Mok S C, Chan W Y, Wong K K, Muto M G, Berkowitz R S. SPARC, an extracellular matrix protein with tumor-suppressing activity in human ovarian epithelial cells. Oncogene. 1996 May 2; 12(9):1895-901
38. Yiu G K, Chan W Y, Ng S W, Chan P S, Cheung K K, Berkowitz R S, Mok S C. SPARC (secreted protein acidic and rich in cysteine) induces apoptosis in ovarian cancer cells. Am J Pathol. 2001 August; 159(2):609-22
39. Said N, Motamed K. Absence of host-secreted protein acidic and rich in cysteine (SPARC) augments peritoneal ovarian carcinomatosis. Am J Pathol. 2005; 167:1739-52
40. Sage H, Vernon R B, Funk S E, Everitt E A, Angello J. Adhesion, shape, proliferation, and gene expression of mouse Leydig cells are influenced by extracellular matrix in vitro. Biol Reprod. 1991 January; 44(1):157-70
41. Chlenski A, Liu S, Guerrero J L, Yang Q, Tian Y, Salwen H R, Zage P, Cohn S L. SPARC expression is associated with impaired tumor growth, inhibited angiogenesis and changes in the extracellular matrix. Int J Cancer. 2006 Jan. 15; 118(2):310-6
42. Chlenski A, Guerrero L J, Yang Q, Tian Y, Peddinti R, Salwen H R, Cohn S L. SPARC enhances tumor stroma formation and prevents fibroblast activation. Oncogene. 2007 Jul. 5; 26(31):4513-22. PMID: 17260013
43. Yang E, Kang H J, Koh K H, Rhee H, Kim N K, Kim H. Frequent inactivation of SPARC by promoter hypermethylation in colon cancers. Int J Cancer. 2007 Aug. 1; 121(3):567-75. PMID: 17397030
44. Ito M, Ito G, Kondo M, Uchiyama M, Fukui T, Mori S, Yoshioka H, Ueda Y, Shimokata K, Sekido Y. Frequent inactivation of RASSF1A, BLU, and SEMA3B on 3p21.3 by promoter hypermethylation and allele loss in non-small cell lung cancer. Cancer Lett. 2005 Jul. 8; 225(1):131-9. PMID: 15922865
45. Suzuki M, Hao C, Takahashi T, Shigematsu H, Shivapurkar N, Sathyanarayana U G, Iizasa T, Fujisawa T, Hiroshima K, Gazdar A F. Aberrant methylation of SPARC in human lung cancers. Br J Cancer. 2005 Mar. 14; 92(5):942-8. PMID: 15756262
46. Soya P, Feng Q, Geiss G, Wood T, Strauss R, Rudolf V, Lieber A, Kiviat N. Discovery of novel methylation biomarkers in cervical carcinoma by global demethylation and microarray analysis. Cancer Epidemiol Biomarkers Prey. 2006 January; 15(1):114-23. PMID: 16434596
47. Watkins G, Douglas-Jones A, Bryce R, Mansel R E, Jiang W G. Increased levels of SPARC (osteonectin) in human breast cancer tissues and its association with clinical outcomes. Prostaglandins Leukot Essent Fatty Acids. 2005 April; 72(4):267-72
48. Kim Y W, Park Y K, Lee J, Ko S W, Yang M H. Expression of osteopontin and osteonectin in breast cancer. J Korean Med Sci. 1998 December; 13(6):652-7. PMID: 9886175
49. Koblinski J E, Kaplan-Singer B R, VanOsdol S J, Wu M, Engbring J A, Wang S, Goldsmith C M, Piper J T, Vostal J G, Harms J F, Welch D R, Kleinman H K. Endogenous osteonectin/SPARC/BM-40 expression inhibits MDA-MB-231 breast cancer cell metastasis. Cancer Res. 2005 Aug. 15; 65(16):7370-7. PMID: 16103089
50. Jones P A, Taylor S M, Wilson V L. Inhibition of DNA methylation by 5-azacytidine. Recent Results Cancer Res. 1983; 84:202-11.
51. Santi D V, Garrett C E, Barr P J. On the mechanism of inhibition of DNA-cytosine methyltransferases by cytosine analogs. Cell. 1983; 33:9-10.
52. Gabbara S, Bhagwat A S. The mechanism of inhibition of DNA (cytosine-5-)-methyltransferases by 5-azacytosine is likely to involve methyl transfer to the inhibitor. Biochem J. 1995; 307:87-92.
53. Bird A P. The relationship of DNA methylation to cancer. Cancer Surv. 1996; 28:87-101.
54. Jones P A, Laird P W. Cancer epigenetics comes of age. Nat Genet. 1999; 21:163-7.
55. Karpf A R, Jones D A. Reactivating the expression of methylation silenced genes in human cancer. Oncogene. 2002; 21(35):5496-503.
56. Uchida T, Kinoshita T, Nagai H, Nakahara Y, Saito H, Hotta T, et al. Hypermethylation of the p15INK4B gene in myelodysplastic syndromes. Blood. 1997; 90(4):1403-9.
57. Herman J G, Latif F, Weng Y, Lerman M I, Zbar B, Liu S, et al. Silencing of the VHL tumor-suppressor gene by DNA methylation in renal carcinoma. Proc Natl Acad Sci USA. 1994; 91(21):9700-4.
58. van der Velden P A, Metzelaar-Blok J A, Bergman W, Monique H, Hurks H, Frants R R, et al. Promoter hypermethylation: a common cause of reduced p16(INK4a) expression in uveal melanoma. Cancer Res. 2001; 61(13):5303-6.
59. Dobrovic A, Simpfendorfer D. Methylation of the BRCA1 gene in sporadic breast cancer. Cancer Res. 1997; 57:3347-50.
60. Hiltunen M O, Alhonen L, Koistinaho J, Myohanen S, Paakkonen M, Marin S, et al. Hypermethylation of the APC (Adenomatous Polyposis Colie) gene promoter region in human colorectal carcinoma. Int J Cancer. 1997; 70:644-8.
61. Merlo A, Herman J G, Mao L, Lee D J, Gabrielson E, Burger P C, et al. 5′ CpG island methylation is associated with transcriptional silencing of the tumour suppressor p16/CDKN2/MTS1 in human cancers. Nat Med. 1995; 1(7):686-92.
62. Herman JG, Jen J, Merlo A, Baylin SB. Hypermethylation-associated inactivation indicates a tumor suppressor role for p15INK4B. Cancer Res. 1996;56(4):722-7. 63. Li L H, Olin E J, Buskirk H H, Reineke L M. Cytotoxicity and mode of action of 5-azacytidine on L1210 leukemia. Cancer Res. 1970; 30(November):2760-9.
64. Li L H, Olin E J, Fraser T J, Bhuyan B K. Phase specificity of 5-azacytidine against mammalian cells in tissue culture. Cancer Res. 1970; 30(November):2770-5.
65. Silverman L R. Targeting hypomethylation of DNA to achieve cellular differentiation in myelodysplastic syndromes (MDS). Oncologist. 2001; 6(suppl 5):8-14.
66. Jones P A, Taylor S M, Wilson V. DNA modification, differentiation, and transformation. The Journal of Experimental Zoology. 1983; 228(2):287-95.
67. Leone G, Teofili L, Voso M T, Liibbert M. DNA methylation and demethylating drugs in myelodysplastic syndromes and secondary leukemias. Haematologica. 2002; 87(12):1324-41.
68. Silverman L R, Holland J F, Demakos E P, Peterson B, Nelson D A, Clamon G, et al. Azacitidine (Aza C) in myelodysplastic syndromes (MDS), CALGB studies 8421 and 8921. [abstract 46]. Ann Hematol 1994; 68(2):A12.
69. Robertson K D, Jones P A. DNA methylation: past, present and future directions. Carcinogenesis. 2000; 21(3):461-7.
70. Plagemann P G, Behrens M, Abraham D. Metabolism and cytotoxicity of 5-azacytidine in cultured Novikoff rat hepatoma and P388 mouse leukemia cells and their enhancement by preincubation with pyrazofurin. Cancer Res. 1978; 38(8):2458-66.
71. Juttermann R, Li E, Jaenisch R. Toxicity of 5-aza-2′-deoxycytidine to mammalian cells is mediated primarily by covalent trapping of DNA methyltransferase rather than DNA demethylation. Proc Natl Acad Sci USA. 1994; 91(25):11797-801.
72. Ueno M, Katayama K, Yasoshima A, Nakayama H, Doi K. 5-Azacytidine (5AzC)-induced histopathological changes in the central nervous system of rat fetuses. Exp Toxic Pathol. 2002; 54:91-6.
73. Glover A B, Leyland-Jones B, Chun H G, Davies B, Hoth D F. Azacitidine: 10 years later. Cancer Treat Rep. 1987; 71(7-8):737-46.
74. Pharmion Corporation. Azacitidine Investigator's Brochure, Edition 5. Boulder, Colo.; Jan. 31, 2007.
75. Marcucci G, Silverman L, Eller M, Lintz L, Beach C L. Bioavailability of azacitidine subcutaneous versus intravenous in patients with the myelodysplastic syndromes. J Clin Pharmacol. 2005; 45(5):597-602.
76. Von Hoff D D, Slavik M, Muggia F M. 5-Azacytidine. A new anticancer drug with effectiveness in acute myelogenous leukemia. Ann Intern Med. 1976; 85(2):237-45.
77. Silverman L R, McKenzie D R, Peterson B L, Holland J F, Backstrom J T, Beach C L, et al. Further analysis of trials with azacitidine in patients with myelodysplastic syndrome: studies 8421, 8921, and 9221 by the cancer and leukemia group B. J Clin Oncol. 2006; 24(24):3895-903.
78. Fenaux P, Mufti G, Santini V, Finelli C, Giagounidis A, Schoch R, et al. Azacitidine (AZA) treatment prolongs overall survival (OS) in higher-risk MDS patients compared with conventional care regimens (CCR): Results of the AZA-001 phase III study [abstract 817]. Blood 2007; 110(11 part 1 of 2):250a.

The above description discloses several compositions and methods of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein including, but not limited to, published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

What is claimed is:

1. A method of treating cancer in a subject in need thereof comprising:
increasing expression of a nucleic acid encoding secreted protein acidic and rich in cysteine (SPARC) protein or the level or activity of SPARC protein or increasing the expression or activity of SPARC protein in a tumor cell of the subject; and
administering an albumin-bound chemotherapeutic agent.

2. The method of claim 1, wherein increasing expression of a nucleic acid encoding secreted protein acidic and rich in cysteine (SPARC) protein or the level or activity of SPARC protein or increasing expression or activity of SPARC protein comprises administering a hypomethlyating agent or a histone deacetylase inhibitor.

3. The method of claim 2, wherein the hypomethlyating agent is selected from the group consisting of azacitidine, and decitabine.

4. The method of claim 2, wherein the inhibitor is selected from the group consisting of vorinostat and valproic acid.

5. The method of claim 1, wherein the albumin-bound chemotherapeutic agent comprises an agent selected from the group consisting of paclitaxel, docetaxel, and rapamycin.

6. The method of claim 1, wherein the albumin-bound chemotherapeutic agent is Abraxane.

7. The method of claim 1, wherein the albumin-bound chemotherapeutic agent is administered weekly.

8. The method of claim 1, wherein the cancer comprises a cancer selected from the group consisting of an advanced solid tumor, a metastatic solid tumor, a lymphoma, ovarian cancer, endometrial cancer, lung cancer, a sarcoma, pancreatic cancer, a leukemia, and breast cancer.

9. The method of claim 1, wherein increasing expression of a nucleic acid encoding secreted protein acidic and rich in cysteine (SPARC) protein or the level or activity of SPARC protein or increasing expression or activity of SPARC protein in a tumor cell of the subject comprises administering at least about 75 mg/m² azacitadine, and administering an albumin-bound chemotherapeutic agent comprises administering at least about 100 mg/m² nab-paclitaxel.

10. The method of claim 9, wherein the nab-paclitaxel is administered weekly.

11. The method of claim 10, wherein the nab-paclitaxel is administered for at least 2 weeks.

12. The method of claim 9, wherein the azacitadine is administered prior to administration of nab-paclitaxel.

13. The method of claim 12, wherein azacitadine is administered daily for an initial period and subsequent to said initial period nab-paclitaxel is administered periodically.

14. The method of claim 13, wherein said initial period is 5 days.

15. The method of claim 14, wherein nab-paclitaxel is administered on days 8, 15, and 22.

16. The method of claim 15, wherein said administration of azacitadine and subsequent administration of nab-paclitaxel is repeated for a plurality of cycles.

17. The method of claim 16, wherein said plurality of cycles is 6 cycles.
